(19) United States
(12) Patent Application Publication
(10) Pub. No.: US 2013/0029869 A1
(43) Pub. Date: Jan. 31, 2013

(54) Molecular Profile For the Diagnosis of Metabolic Myopathies

(71) Applicants: Georgirene D. Vladutiu, Williamsville, NY (US); Paul J. Isackson, East Aurora, NY (US)

(72) Inventors: Georgirene D. Vladutiu, Williamsville, NY (US); Paul J. Isackson, East Aurora, NY (US)

(21) Appl. No.: 13/557,762

(22) Filed: Jul. 25, 2012

Related U.S. Application Data
(60) Provisional application No. 61/511,740, filed on Jul. 26, 2011.

(51) Int. Cl. C12Q 1/68; C40B 30/04

(52) U.S. Cl. 506/9; 435/6.12; 435/6.11

(57) ABSTRACT

Provided is a method for determining whether an individual is at risk for, or has a metabolic muscle disease. The method involves testing a biological sample obtained or derived from an indivdival for the presence or absence of at least one mutation from a plurality of mutations in genes related to muscle metabolism and identifying the individual as having or at risk for developing a metabolic muscle disease based on the presence or absence of the mutation.

### CROSS-REFERENCE TO RELATED APPLICATIONS

The present invention claims priority to U.S. provisional patent application No. 61/511,740, filed Jul. 26, 2012, the disclosure of which is incorporated herein by reference.

### STATEMENT REGARDING FEDERALLY SPONSORED RESEARCH

This invention was made with government support under RO1-HL085800 and R41-RHL093956 awarded by National Institutes of Health. The government has certain rights in the invention.

### FIELD OF THE INVENTION

The present invention relates generally to the field of muscle disorders and more specifically to a panel of markers that is useful for diagnosis of muscle disorders and for developing a personalized treatment plan for individuals diagnosed with or at risk for developing muscle disorders.

### BACKGROUND OF THE INVENTION

Metabolic muscle disease resulting from exposure to environmental triggers including statin therapy, anesthesia, viral infection, temperature extremes, exertion, fasting, stress and sleep deprivation result in myopathy and life-threatening rhabdomyolysis in greater than 200,000 people in the USA per year. There is a great need for (1) improved diagnosis of patients with metabolic muscle disease for whom overlapping symptoms complicate diagnosis, and for whom detailed invasive muscle biopsies and biochemical tests are required for proper diagnosis and therapy; and (2) preventive determination of risk for adverse reactions to statins, anesthesia and stressful environmental factors. The present invention meets these and other needs.

### SUMMARY OF THE INVENTION

The present invention provides a method for determining whether an individual is at risk for, or has a metabolic muscle disease. The method comprises testing a biological sample obtained or derived from the indivdival for the presence or absence of at least one mutation from a plurality of mutations as further described herein. In various embodiments, the invention includes determining homozygosity and heterozygosity of an indivdival for one or a combination of mutations disclosed herein. The mutations include those in List 1, List 2 and List 3 presented below. In one aspect of the invention, the method comprises identifying an individual as having or being at risk for developing a metabolic myopathy, such as a metabolic myopathy related to Carnitine palmitoyltransferase II deficiency, by testing for the presence or absence of one or a combination of the CPT2 mutations disclosed herein. CPT2 encodes Carnitine palmitoyltransferase II.

In another aspect of the invention, the method comprises identifying an individual as having or being at risk for developing metabolic myopathies related to altered glycogen storage by testing for the presence or absence of one or a combination of the PYGM mutations disclosed herein. PYGM encodes myophosphorylase and mutations in it can affect glycogen catabolism.

### DESCRIPTION OF THE INVENTION

We have developed a microarray approach using polynucleotide arrary technology for screening for multiple metabolic muscle diseases induced by various triggers including but not necessarily limited to statins, anesthesia, exertion, fasting, temperature extremes, viral infection, stress and sleep deprivation. A metabolic muscle disorder is considered to be a muscular disorder that is caused by impairment of a metabolic pathway involved in energy production or utilization.

In general, the invention provides a method of assessing pathologic genotype—phenotype correlations using genetic determinants and clinical correlates to (a) determine an individual's diagnosis of metabolic muscle disease and/or (b) an individual's risk to develop muscle injury or a muscle disease in the future.

The invention relates to certain environmental factors, referred to herein as ‘triggers’, which contribute to an individual's risk for having or developing muscle injury or muscle disease. It is considered that these triggers include but are not necessarily limited to exposure to strenuous exertion, fasting, sleep deprivation, stress, extremes in temperature, viral infection, and certain medications such as general anesthesia, cholesterol-lowering drugs, and drugs used to treat certain psychiatric illnesses. In one embodiment, the invention is particularly suited for detecting high risk individuals and developing a profile for them so that they can avoid becoming ill due to statin therapy or anesthesia.

The invention utilizes an ensemble of mutations that are positively correlated with development of, or risk for developing muscle disease, together with individual clinical characteristics and laboratory findings, to determine either a diagnosis of or a risk for muscle disease for an individual. In one embodiment, the invention includes developing individual genotype-phenotype scores which will result in a weighted diagnostic score or a disease risk score for an individual. The scores can be provided on a report that will include the score, its interpretation, and an interpretation of the test results.

In one embodiment, the method of the invention comprises determining from a genetic sample obtained or derived from an individual the presence or absence of one or more mutations selected from the group (an ensemble) of mutations presented in List 1 and/or List 2 and/or List 3. The presence or absence of the one or more mutations can be used to develop a pathologic genomic profile for the individual. The pathologic genomic profile can be correlaetd with the phenotype of the individual by comparing the individual's genomic mutation profile and phenotypic features with a database of human genotype-phenotype correlations that includes somoe or all the members of the ensemble of mutations. The invention also contemplates developing a profile of novel genotype-phenotype correlations comprising the unique phenotypic characteristics associated with the presence of one or more of the ensemble of mutations associated with one or more diseases found in an individual and noting that the individual's phenotype may represent different characteristics than usually expected for the mutation(s). Based on the presence or absence of the one or more mutations in the ensemble, the invention provides for determining a diagnosis of muscle disease or risk for developing muscle disease based on genotype-phenotype correlations. Also provided is a method of assembling the results of the genetic phenotype-genotype correlations and developing and providing reports of test results to a health care provider for the individual. The invention further comprises updating one or more databases of human genotype correlations with additional novel human genotype-phenotype correlations as the additional correlations become known due to practicing the method of the invention.

In one embodiment, determining that an individual is at risk for or has a metabolic muscle diseases is indicative that the indivdival should not receive a statin, or should receive a reduced dose or altered dosing shedule of the statin. In this embodiment, the method can further comprise altering the health care plan of the individual to compensate for a recommendation that the statin treatment regime be altered due to the presence of the mutation.

One embodiment of the invention provides an ensemble of target genes useful for (a) determining an individual's diagnosis of metabolic muscle disease or (b) an individual's risk to develop muscle injury or a muscle disease in the future and in response to selected triggers. The method compriseses determining at least one detrimental gene mutation within the ensemble of target genes, the presence of which correlates with at least one injury to muscle or muscle side effects in humans, wherein said injury selected from the group comprised of serum creatine kinase activity greater than three times the upper limit of normal for gender and race of the individual; the presence of abnormal elevations of myoglobin in urine; severe muscle pain, cramps, stiffness, and/or muscle weakness; abnormal elevation of body temperature above 39 degrees centigrade; other markers of muscle injury including abnormal (high or low) levels of serum lactate and ammonia or myopathic abnormalities detected in histochemical assessment of muscle biopsy in an individual with muscle injury or disease; and combinations thereof. The ensemble of target genes can be comprised of one, or more than one detrimental mutation per gene associated with muscle injury and/or muscular side effects in response to one or more trigger exposures, wherein the genetic mutation or mutations are within the target genes selected from the group the following group. Each of these genes is known in the art and its nucleotide sequence is designated by an NCBI database number as follows. The nucleotide and/or amino acid sequence associated with each NCBI database number shown is incorporated herein as it exists in the database as of the filing date of this application. Each gene name and its databased entry is followed by the name of at least one muscle disorder to which the gene is known or thought to be related. The genes in which mutations in the ensemble of mutations disclosed herein arise are selected from: ACAD9 (NM_{—}014049, Complex I deficiency), AMPD1 (NM_{—}000036, Myoadenylate deficiency), CACNA1S (NM_{—}000069, malignant hyperthermia), CAV3 (NM_{—}033337, rippling muscle disease, hyperCKemia, limb girdle muscular dystrophy), CPT2 (NM_{—}000098, CPTII deficiency), ETFA (NM_{—}000126, ETF deficiency), ETFB (NM_{—}001985, ETF deficiency), ETFDH (NM_{—}004453, multiple acyl-CoA dehydrogenase deficiency), HADHA (NM_{—}000182, mitochondrial trifunctional protein deficiency), HADHB (NM_{—}000183, mitochondrial trifunctional protein deficiency), LPIN1 (NM_{—}145693, hyperCKemia), ACADM (NM_{—}000016, medium-chain acyl-CoA dehydrogenase deficiency), PTRF (NM_{—}012232, lipodystrophy), PYGM (NM_{—}001164716, McArdle disease), RYR1 (NM_{—}000540, malignant hyperthermia) and ACADVL (NM_{—}000018, very-long chain acyl-CoA dehydrogenase deficiency) and combinations thereof.

It will be apparent to one skilled in the art that this invention encompasses virtually every method by which the presence (or absence) of the one or more mutations from the ensemble of mutations could be detected. In one aspect of the invention, the presence (or absence) of the one or more mutations in the ensemble can be detected directly. Direct detection can be performed, for example, by detecting the mutation by using DNA or RNA sequencing, PCR, QPCR, in situ PCR, primed in situ hybridization, or Southern Hybridization. Detection could also be performed by hybridization or in situ hybridization using one or more probes, such as by a gene array, SNP chip, or any other suitable method and/or device for detecting the presence of one or more nucleic acid biomarkers.

In one embodiment, the presence or absence of the mutations are determined by analyzing nucleic acids obtained or derived from an individual using a polynucleotide array presented on a chip. Chips suitable for use in the present invention can be designed and made using known techniques and/or obtained from a variety of commercial chip vendors, such as Affymetrix. For certain data presented herein, we used commercially available genetic screening technology available from SEQUENOM (San Diego, Calif.). In one embodiment, a chip design will provide for assaying a plurality of mutations that include at least 2-500 mutations, inclusive, and including all digits there between. In one embodiment, the chip design will provide for assaying 360 mutations selected from List 1 and/or List 2, and/or mutations that come to be known as correlated with a muscle disease phenotype by operation of the instant invention or otherwise.

The nucleic acid sample obtained from or derived from the individual can be DNA and/or RNA, and can be analyzed to determine whether the individual has or is at risk for any of a wide variety of muscle diseases.

The presence or absesence of the one or more mutations from the ensemble of mutations disclosed herein can also be indirectly detected. Indirect detection could be performed, for example, by detecting protein expressed by a gene comprising the mutation in a sample, for example by immunodetection or immunoassay (such as Western Blot, ELISA, florescent labeling, radioimmunoassay, secretion assay, or immunostaining), protein labeling (for example with visual or flow cytometry detection), or other non-immunoassays known to those skilled in the art.

The presence or absence of the one or more mutations could also be indirectly detected by assaying the levels of metabolic byproducts which result when the gene comprising the one or more mutation is expressed. For example, differences in cell secretions due to altered biochemical pathways affected by mutated gene expression could be detected by a tissue, blood, or urine test. Alternatively, alterations of cell surface expressed proteins could be detected, for example by antibody labeling and flow cytometry.

In one embodiment, a DNA sample is analyized on a chip for one or more mutations, the presence or absence of which is correlated with at least two muscle diseases. In certain embodiments, a plurality of mutations are analyzed for a correlation with at least 2, 3, 4, 5, 6, 7, 8, 9, 10, 11, 12, 13, or more muscle diseases. In one embodiment, up to 400 mutations are analyzed to determine whether an individual has or is at risk for 11, 12 or 13 metabolic muscle disorders.

In another aspect of the invention, the presence or absence of the mutations can be detected in a variety of different biological samples or in preparations derived from biological samples. For example, the mutations could be detected from a sample taken as part of a muscle biopsy. However, since the mutations are inherited, their presence or absence can be determined in a sample taken or derived from any other part of the body. For example, the determination could be performed using a saliva, blood, urine, hair, skin, buccal swab, or any other sample that contains nucleic acids. The sample can be taken from the individual using any of the methods which are known in the art for taking tissue or other biological samples from a patient. The sample could also be taken from a culture of tissue which has been grown from a biological sample taken from a patient. For example, the culture could be an in vitro culture. The culture could also comprise immortalized cells derived from a tissue taken from the patient. Thus, it will be apparent to one skilled in the art that this invention encompasses virtually every method by which a genetic sample might be acquired and/or amplified and/or derived from a patient and tested for the presence (or absence) of the one or more mutations. Further, the method is suitable for use with samples from an adults, children and infants. The method is intended for use with humans, but could also be applicable to non-human animals which share mutations in the genes described herein.

In one embodiment, it is considered that the absence of the one or more mutation is indicative of an a lower risk of developing one or more muscle disorders relative to the prognosis of an individual wherein the presence of the one or more mutation is detected.

In certain aspects of the invention, the method comprises identifying an individual as having or being at risk for developing a metabolic myopathy, such as a metabolic myopathy related to Carnitine palmitoyltransferase II deficiency. This aspect involves testing for the presence or absence of one or a combination of the CPT2 mutations disclosed herein. CPT2 encodes Carnitine palmitoyltransferase II. Mutations in it can lead to a disorder of long-chain fatty-acid oxidation which result in three clinical presentations: Lethal Neonatal form of CPT2 deficiency, Severe Infantile Hepatocardiomuscular form of CPT2 deficiency and the Mild Adult Onset Myopathic form of CPT2 deficiency. In one embodiment, identifying an individual as having or likely to develop CPT2 deficiency comprises testing a biological sample from the individual for the presence or absence of at least one mutation selected from the group of CPT2 mutations described in List 1, List 2, List 3, or combinations of those CPT2 mutations.

In another aspects of the invention, the method comprises identifying an individual as having or being at risk for developing metabolic myopathies related to glycogen storage, such as glycogen storage disease type, a non-limiting examples of which includes Glycogen storage disease type V or McArdle disease. This aspect involves testing for the presence or absence of one or a combination of the PYGM mutations disclosed herein. PYGM encodes myophosphorylase and mutations in it can affect glycogen catabolism. In one embodiment, identifying an individual as having or likely to develop a metabolic myopathy, such as a metabolic myopathy related to glycogen storage comprises testing a biological sample from the individual for the presence or absence of at least one mutation selected from the group of PYGM mutations described in List 1, List 2, List 3, or combinations of those PYGM mutations.

The ensemble of disease-causing mutations in genes relevant to muscle disease for determining whether an individual has or is at risk for devloping a muscle disease or muscle injury in response to environmental triggers is set forth in List 1, List 2 and List 3. In these lists, the mutation description nomenclature is presented as recommended by the human genome variation society. This nomeclature is understood by those skilled in the art and can be accesses at: www.hgvs.org/mutnomen/). To illsutrate, “ACAD9_F44I_{—}130T>A” means there is a change of the amino acid at position 44 and a change of the nucleotide at position 130 in the wild type ACAD gene. Where designated, such as in List 2 and List 3, “p.” refers to the amino acid sequence position and “c.” refers to the cDNA nucleotide position where numbering begins with the start codon. In List 1, the first sequence following the gene name is the amino acid sequence variation and the second sequence is the cDNA nucleotide position. In List 3, PolyPhen2 is a predictive algorithm known in the art that uses both sequence alignment of homologous proteins and structure-based prediction algorithms (see, Adzhubei I A, et al. Nat Methods 7(4):248-249 (2010)). We identified the mutations that are accorded PolyPhen2 scores from myopathic CPT2 patient samples

### List 1

ACAD9_F44I_{—}130T>A, ACAD9_R127Q_{—}379A>C/380G>A, ACAD9_R226Q_{—}797G>A, ACAD9_R417C_{—}1249C>T, ACAD9_R469W_{—}1405C>T, ACAD9_R532W_{—}1594C>T, AMPD 1_Q12X_{—}34C>T, AMPD 1_ivs2_{—}35-(4_{—}7)delCTTT, AMPD1_P48L_{—}143C>T, AMPD1_Q156H_{—}468G>T, AMPD1_K287I_{—}860A>T, AMPD1_M310I_{—}930G>T, CACNA1S_R174W_{—}520C>T, CACNA1S_R1086S_{—}3256C>A, CACNA1S_R1086H_{—}3257G>A, CACNA1S_T1354S_{—}4060A>T, CAV3_R27Q_{—}80G>A, CAV3_D28E_{—}84C>A, CAV3_P29T_{—}85C>A, CAV3_P29L_{—}86C>T, CAV3_N33K_{—}99C>G, CAV3_E34K_{—}100G>A, CAV3_ivs1_{—}114+2T>C, CAV3_V44E_{—}131T>A, CAV3_A46T_{—}136G>A, CAV3_A46V_{—}137C>T, CAV3_E47A_{—}140A>C, CAV3_S53G_{—}157A>G, CAV3_G56S_{—}166G>A, CAV3_V57M_{—}169G>A, CAV3_S61R_{—}183C>A, CAV3_delT64F65T66_{—}189_{—}197delCACCTTCAC, CAV3_T64P_{—}190A>C, CAV3_T64S_{—}191C>G, CAV3_W71X_{—}212G>A, CAV3_C72W_{—}216C>G, CAV3_Y73C_{—}218A>G, CAV3_T78K_{—}233C>A, CAV3_T78M_{—}233C>T, CAV3_L79R_{—}236T>G, CAV3_A85T_{—}253G>A, CAV3_L87P_{—}260T>C, CAV3_A93T_{—}277G>A, CAV3_delF97_{—}289-291delTTC, CAV3_F97C_{—}290T>G, CAV3_W101R_{—}301T>C, CAV3_delL103L104_{—}307-312delGTGGTG, CAV3_P105L_{—}314C>T, CAV3_S141R_{—}423C>G, CPT2_L7fs_{—}20-21insT, CPT2G13fs_{—}35-38delG, CPT2A15fs_{—}36-43dupGGGCCCCG, CPT2_Q33fs_{—}98delA, CPT2S38fs_{—}113-114dupGC, CPT2P50H_{—}149C>A, CPT2P55R_{—}164C>G, CPT2T6ON_{—}179C>A, CPT2_ivs2_{—}233+1G>C, CPT2_ivs2_{—}234−1G>A, CPT2C84R_{—}250T>C, CPT2_S86fs_{—}254-257delAG, CPT2S113L_{—}338C>T, CPT2_ivs3_{—}345+5G>A, CPT2Y120C_{—}359A>G, CPT2R124X_{—}370C>T, CPT2R124Q_{—}371G>A, CPT2R151Q_{—}452G>A, CPT2R161W_{—}481C>T, CPT2K164X_{—}490A>T, CPT2_P173S_{—}517C>T, CPT2E174K_{—}520G>A, CPT2L178-I186delinsF_{—}534-558delinsT, CPT2Y210D_{—}628T>G, CPT2D213G_{—}638A>G, CPT2M214T_{—}641T>C, CPT2P227L_{—}680C>T, CPT2P227R_{—}680C>G, CPT2R231W_{—}691C>T, CPT2N250fs_{—}747-749delAA, CPT2_S267L_{—}800C>T, CPT2K274M_{—}821A>T, CPT2P284fs_{—}848-852delC, CPT2R296X_{—}886C>T, CPT2_R296Q_{—}887G>A, CPT2_R296L_{—}887G>T, CPT2_C324Y_{—}971G>A, CPT2_C326Y_{—}977G>A, CPT2D328G_{—}983A>G, CPT2_M342T_{—}1025T>C, CPT2_R350C_{—}1048C>T, CPT2_F352C_{—}1055T>G, CPT2_A367D_{—}1100C>A, CPT2_F383Y_{—}1148T>A, CPT2_S408fs_{—}1221-1224delCT, CPT2Q413fs_{—}1238-1239delAG, CPT2_K414fs_{—}1239-1240delGA, CPT2_T425fs_{—}1273-1274delAC, CPT2_L441fs_{—}1323_{—}1326delCACT, CPT2_F448L_{—}1342T>C, CPT2_R450X_{—}1348A>T, CPT2_E454X_{—}1360G>T, CPT2_K457X_{—}1369A>T, CPT2_K458Q_{—}1372A>C, CPT2_Q472X_{—}1414C>T, CPT2_Y479C_{—}1436A>G, CPT2_G480R_{—}1438G>A, CPT2_T482fs_{—}1444-1447delACAG, CPT2_E487K_{—}1459G>A, CPT2_I502T_{—}1505T>C, CPT2_R503C_{—}1507C>T, CPT2_P504L_{—}1511C>T, CPT2_C512Y_{—}1535G>A, CPT2_A515fs_{—}1543-1546delGCCT, CPT2_F516S_{—}1547T>C, CPT2_H523fs_{—}1567-1570delCA, CPT2_E545del_{—}1631-1636delAAG, CPT2_ivs4_{—}1645+5G>A, CPT2_G549D_{—}1646G>A, CPT2_Q550R_{—}1649A>G, CPT2_D553N_{—}1657G>A, CPT2_R554X_{—}1660C>T, CPT2_R560Q_{—}1679G>A, CPT2_Y579fs_{—}1737delC, CPT2_S588C_{—}1763C>G, CPT2_T589fs_{—}1767delG, CPT2_P595fs_{—}1782-1784delC, CPT2_G600R_{—}1798G>A, CPT2_G601R_{—}1801G>C, CPT2_P604S_{—}1810C>T, CPT2_P604L_{—}1811C>T, CPT2_V605L_{—}1813G>C, CPT2_V606fs_{—}1816-1817delGT, CPT2_D608H_{—}1822G>C, CPT2_Y628S_{—}1883A>C, CPT2_R631C_{—}1891C>T, CPT2_E641fs_{—}1 923-1935del13, CPT2_E645fs_{—}1932-1933insA, CPT2_E645fs_{—}1933-1934insG, ETFA_R3X_{—}7C>T, ETFA_Q9fs_{—}12-22dup, ETFA_R18X_{—}52C>T, ETFA_I25fs_{—}72-73delA, ETFA_L95W_{—}284T>G, ETFA_G116R_{—}346G>A, ETFA_L119fs_{—}354-356insC, ETFA_R122K_{—}365G>A, ETFA_F144S_{—}431T>C, ETFA_V157G_{—}470T>G, ETFA_D160fs_{—}478delG, ETFA_V165A_{—}494T>C, ETFA_ivs6_{—}563-1G>C, ETFA_L212P_{—}635T>C, ETFA_R249C_{—}745C>T, ETFA_G255V_{—}764G>T, ETFA_T266M_{—}797C>T, ETFA_G267R_{—}799G>A, ETFA_delV270_{—}806-811del3, ETFA_D292fs_{—}875delA, ETFB_K19X_{—}55A>T, ETFB_R26fs_{—}77-78delG, ETFB_T27fs_{—}80-81delC, ETFB_C42R_{—}124T>C, ETFB_ivs3_{—}376-1G>C, ETFB_D128N_{—}382G>A, ETFB_R164Q_{—}491G>A, ETFB_R191C_{—}571C>T, ETFB_ivs5_{—}597+1G>C, ETFB_delK206_{—}607-618delAAG, ETFDH_L377P_{—}1130T>C, ETFDH_P456L_{—}1367C>T, ETFDH_P483L_{—}1448C>T, ETFDH_K590E_{—}1768A>G, HADHA_R676H_{—}2027G>A, HADHA_E510Q_{—}1528G>C, HADHA_ivs9_{—}919-2A>G, HADHA_A244V_{—}731C>T, HADHB_R61C_{—}181C>T, HADHB_N114S_{—}341A>G, HADHB_L121P_{—}362T>A, HADHB_N142K_{—}426C>A, HADHB_S176X_{—}527C>G, HADHB_R203X_{—}607C>T, HADHB_D242G_{—}725A>G, HADHB_D263G_{—}788A>G, HADHB_G301D_{—}902G>A, HADHB_A459E_{—}1376C>A, LPIN1_Y19X_{—}57C>A, LPIN1_R388X_{—}1162C>T, LPIN1_ivs9_{—}1441+2T>C, LPIN1_P420fs_{—}1258-1259delC, LPIN1_L752fs_{—}2251-2254delCC, LPIN1_E769G_{—}2306A>G, LPIN1_R801X_{—}2401C>T, LPIN1_ivs19_{—}2513+1G>A, ACADM_R53C_{—}157C>T, ACADM_I78T_{—}233T>C, ACADM_G267R_{—}799G>A, ACADM_K329E_{—}985A>G, PTRF_{—}159-160delG, PTRF_{—}362dupT, PTRF_{—}512C>A, PTRF_E176fs_{—}525-526delG, PTRF_{—K}233fs_{—}696dupC, PYGM_M1L_{—}1A>C, PYGM_M1V_{—}1A>G, PYGM_L5fs_{—}13-14delCT, PYGM_V16fs_{—}46insTTdelG, PYGM_T26fs_{—}78-79delTG, PYGM_R50X_{—}148C>T, PYGM_Y53X_{—}159C>G, PYGM_Q73fs_{—}212-218dup, PYGM_I83F_{—}247A>T, PYGM_Y85X_{—}255 C>A, PYGM_R94W_{—}280C>T, PYGM_N102fs_{—}304-305delA, PYGM_L116P_{—}347T>C, PYGM_E125X_{—}373G>T, PYGM_N134fs_{—}402delC, PYGM_G136fs_{—}403-408insG, PYGM_G136D_{—}407G>A, PYGM_R139W_{—}415C>T, PYGM_G157V_{—}470G>T, PYGM_G159R_{—}475G>A, PYGM_R161C_{—}481C>T, PYGM_K170del_{—}506-511del3, PYGM_G174D_{—}521G>A, PYGM_R194W_{—}580C>T, PYGM_G205S_{—}613G>A, PYGM_P230R_{—}689C>G, PYGM_V239del_{—}715-717delGTC, PYGM_ivs6_{—}773-2A>T, PYGM_R270X_{—}808C>T, PYGM_ivs7_{—}855+1G>C, PYGM_L292P_{—}875T>C, PYGM_Q337R_{—}1010A>G, PYGM_E349K_{—}1045G>A, PYGM_E349X_{—}1045G>T, PYGM_W362X_{—}1085G>A, PYGM_ivs9_{—}1092+1G>A, PYGM_ivs9_{—}1093-1G>T, PYGM_A365E_{—}1094C>A, PYGM_A365V_{—}1094C>T, PYGM_T379M_{—}1136C>T, PYGM_E383K_{—}1147G>A, PYGM_A384D_{—}1151C>A, PYGM_L385fs_{—}1155-1156delGG, PYGM_W388fs_{—}1162insAdel8, PYGM_L397P_{—}1190T>C, PYGM_ivs10_{—}1239+1G>A, PYGM_R428C_{—}1282C>T, PYGM_G449R_{—}1345G>A, PYGM_S450L_{—}1349C>T, PYGM_A452fs_{—}1354insC, PYGM_V456M_{—}1363G>A, PYGM_G486D_{—}1457G>A, PYGM_T488N_{—}1463C>A, PYGM_T488I_{—}1463C>T, PYGM_R490W_{—}1468C>T, PYGM_R490Q_{—}1469G>A, PYGM_R491Afs_{—}1469-1470dupG, PYGM_R491C_{—}1471C>T, PYGM_W492X_{—}1475G>A, PYGM_V494fs_{—}1479-1480delG, PYGM_D511fs_{—}1530-1531delG, PYGM_D534fs_{—}1601delA, PYGM_E541X_{—}1621G>T, PYGM_K543X_{—}1627A>T, PYGM_K543T_{—}1628A>C, PYGM_R570W_{—}1708C>T, PYGM_R570Q_{—}1709G>A, PYGM_Y574X_{—}1722T>G, PYGM_K575E_{—}1723A>G, PYGM_R576X_{—}1726C>T, PYGM_Q577R_{—}1730A>G, PYGM_L587P_{—}1760T>C, PYGM_ivs14_{—}1768+1G>A, PYGM_R590H_{—}1769G>A, PYGM_F599fs_{—}1792-1797delT, PYGM_R602W_{—}1804C>T, PYGM_R602Q_{—}1805G>A, PYGM_ivs15_{—}1828-1G>A, PYGM_R650X_{—}1948C>T, PYGM_E655K_{—}1963G>A, PYGM_A660D_{—}1979C>A, PYGM_D662A_{—}1985A>C, PYGM_Q666E_{—}1996C>G, PYGM_N685Y_{—}2053A>T, PYGM_G686R_{—}2056G>A, PYGM_G686R_{—}2056G>C, PYGM_A687P_{—}2059G>C, PYGM_T692fs_{—}2075insAAAdelCC, PYGM_A704V_{—}2111C>T, PYGM_G705fs_{—}2112-2114delGG, PYGM_F709-F710del_{—}2125-2130delTTC, PYGM_R715W_{—}2143C>T, PYGM_K754fs_{—}2260-2262delA, PYGM_Q755X_{—}2263C>T, PYGM_ivs18_{—}2312+3G>C, PYGM_C784X_{—}2352C>A, PYGM_ivs19_{—}2380-1G>A, PYGM_P795fs_{—}2385-2386delAA, PYGM_W798R_{—}2392T>C, PYGM_D815A_{—}2444A>C, PYGM_W826S_{—}2477G>C, RYR1_L13R_{—}38T>G, RYR1_C35R_{—}103T>C, RYR1_R44C_{—}130C>T, RYR1_C64R_{—}190T>C, RYR1_R163C_{—}487C>T, RYR1_R163L_{—}488G>T, RYR1_E209K_{—}625G>A, RYR1_M226K_{—}677T>A, RYR1_G248R_{—}742G>A, RYR1_G341R_{—}1021G>A, RYR1_R367L_{—}1100G>T, RYR1_I403M_{—}1209C>G, RYR1_Y522S_{—}1565A>C, RYR1_R533C_{—}1597C>T, RYR1_D544Y_{—}1630G>T, RYR1_R552W_{—}1654C>T, RYR1_R614C_{—}1840C>T, RYR1_R614L_{—}1841G>T, RYR1_R1043C_{—}3127C>T, RYR1_D1056N_{—}3166G>A, RYR1_R1127H_{—}3380G>A, RYR1_K1467R_{—}4400A>G, RYR1_I1571V_{—}4711A>G, RYR1_R1667C_{—}4999C>T, RYR1_K2013Q_{—}6037A>C, RYR1_R2126Q_{—}6377G>A, RYR1_R2163C_{—}6487C>T, RYR1_R2163H_{—}6488G>A, RYR1_R2163P_{—}6488G>C, RYR1_V2168M_{—}6502G>A, RYR1_A2200V_{—}6599C>T, RYR1_T2206R_{—}6617C>G, RYR1_T2206M_{—}6617C>T, RYR1_N2283H_{—}6847A>C, RYR1_R2336H_{—}7007G>A, RYR1_N2342S_{—}7025A>G, RYR1_A2350T_{—}7048G>A, RYR1_G2375A_{—}7124G>C, RYR1_D2400G_{—}7199A>G, RYR1_A2428T_{—}7282G>A, RYR1_G2434R_{—}7300G>A, RYR1_R2435H_{—}7304G>A, RYR1_R2435L_{—}7304G>T, RYR1_R2454C_{—}7360C>T, RYR1_R2454H_{—}7361G>A, RYR1_R2458C_{—}7372C>T, RYR1_R2458H_{—}7373G>A, RYR1_R2508C_{—}7522C>T, RYR1_R2593G_{—}7777C>G, RYR1_D2730G_{—}8189A>G, RYR1_S2843P_{—}8527T>C, RYR1_W3284R_{—}9850T>A, RYR1_P3410Q_{—}10229C>A, RYR1_D3501Y_{—}10501G>T, RYR1_R3772W_{—}11314C>T, RYR1_R3772Q_{—}11315G>A, RYR1_R3903Q_{—}11708G>A, RYR1_Y3933C_{—}11798A>G, RYR1_G3938D_{—}11813G>A, RYR1_G4104R_{—}12310G>C, RYR1_Y4796C_{—}14387A>G, RYR1_F4808N_{—}14422T>A,14423T>A, RYR1_T4826I_{—}14477C>T, RYR1_L4838V_{—}14512C>G, RYR1_V4842M_{—}14524G>A, RYR1_V4847L_{—}14539G>C, RYR1_R4861II_{—}14582G>A, RYR1_I4898T_{—}14693T>C, RYR1_I4928V_{—}14782A>G, RYR1_G4935S_{—}14803G>A, RYR1_P4973L_{—}14918C>T, RYR1_M4990V_{—}14968A>G, ACADVL_ivs1_{—}62+1G>A, ACADVL_Q13X_{—}37C>T, ACADVL_S22X_{—}65C>A, ACADVL_P35fs_{—}102-104delC, ACADVL_G76E_{—}227G>A, ACADVL_T84fs_{—}249-252del2, ACADVL_P89S_{—}265C>T, ACADVL_P91Q_{—}272C>A, ACADVL_Q100fs_{—}295-299del2, ACADVL_ivs5_{—}341-1delG, ACADVL_N122D_{—}364A>G, ACADVL_delE129_{—}384-391del3, ACADVL_W133X_{—}398G>A, ACADVL_Q145X_{—}433C>T, ACADVL_T158N_{—}473C>A, ACADVL_Q149R_{—}476A>G, ACADVL_A161T_{—}481G>A, ACADVL_V164G_{—}491T>G, ACADVL_V174M_{—}520G>A, ACADVL_G185S_{—}553G>A, ACADVL_G193R_{—}577G>A, ACADVL_G193R_{—}577G>C, ACADVL_Y201C_{—}602A>G, ACADVL_L205P_{—}614T>C, ACADVL_ivs7_{—}623-2A>C, ACADVL_ivs7_{—}623-1G>A, ACADVL_A213T_{—}637G>A, ACADVL_A213P_{—}637G>C, ACADVL_C215fs_{—}644-647delGTCT, ACADVL_E218K_{—}652G>A, ACADVL_G222R_{—}664G>A, ACADVL_R229X_{—}685C>T, ACADVL_P236fs_{—}708-709delCT, ACADVL_C237fs_{—}710-711delGT, ACADVL_L243R_{—}728T>G, ACADVL_K247Q_{—}739A>C, ACADVL_K247E_{—}739A>G, ACADVL_K247T_{—}740A>C, ACADVL_ivs8_{—}753-11T>G, ACADVL_ivs8_{—}753-2A>C, ACADVL_T260M_{—}779C>T, ACADVL_K264E_{—}790A>G, ACADVL_V267fs_{—}799-802delGTTA, ACADVL_delE277_{—}827-831del3, ACADVL_delK278_{—}830-835del3, ACADVL_A281D_{—}842C>A, ACADVL_V283A_{—}848T>C, ACADVL_R286G_{—}856A>G, ACADVL_G289R_{—}865G>A, ACADVL_G290D_{—}869G>A, ACADVL_G294E_{—}881G>A, ACADVL_G294fs_{—}881-887del7, ACADVL_P296fs_{—}887-888delCT, ACADVL_P296fs_{—}888-889delTG, ACADVL_delK298_{—}890-895del3, ACADVL_K299M_{—}896A>T, ACADVL_K299N_{—}897G>T, ACADVL_A304T_{—}910G>A, ACADVL_T307fs_{—}917-921del2, ACADVL_F311fs_{—}931-932delT, ACADVL_V317A_{—}950T>C, ACADVL_P318L_{—}953C>T, ACADVL_A333fs_{—}995-997insT, ACADVL_M352V_{—}1054A>G, ACADVL_ivs10_{—}1077+2T>C, ACADVL_R366C_{—}1096C>T, ACADVL_R366H_{—}1097G>A, ACADVL_I373F_{—}1117A>T, ACADVL_delE381_{—}1139-1143del3, ACADVL_K382Q_{—}1144A>C, ACADVL_R385W_{—}1153C>T, ACADVL_Y391C_{—}1172A>G, ACADVL_ivs11_{—}1182+1G>A, ACADVL_ivs11_{—}1183-15A>G, ACADVL_Y398X_{—}1194C>A, ACADVL_D405H_{—}1213G>C, ACADVL_T409M_{—}1226C>T, ACADVL_I413R_{—}1238T>G, ACADVL_A415fs_{—}1245-1256del12, ACADVL_A416T_{—}1246G>A, ACADVL_S423S_{—}1269G>A, ACADVL_W427X_{—}1280G>A, ACADVL_G439D_{—}1316G>A, ACADVL_G441D_{—}1322G>A, ACADVL_G447R_{—}1339G>A, ACADVL_R450H_{—}1349G>A, ACADVL_R453Q_{—}1358G>A, ACADVL_D454N_{—}1360G>A, ACADVL_R456H_{—}1367G>A, ACADVL_F458L_{—}1372T>C, ACADVL_R459W_{—}1375C>T, ACADVL_E462K_{—}1384G>A, ACADVL_G463E_{—}1388G>A, ACADVL_R469W_{—}1405C>T, ACADVL_R469Q_{—}1406G>A, ACADVL_C477Y_{—}1430G>A, ACADVL_G481D_{—}1442G>A, ACADVL_A490P_{—}1468G>C, ACADVL_delL500_{—}1497-1502del3, ACADVL_L502Q_{—}1505T>A, ACADVL_E504D_{—}1512G>T, ACADVL_R511W_{—}1531C>T, ACADVL_ivs16_{—}1606-(2-3)delAA, ACADVL_ivs17_{—}1679-6G>A, ACADVL_M601fs_{—}1798-1801delA, ACADVL_R613W_{—}1837C>T, ACADVL_R615X_{—}1843C>T, ACADVL_A640fs_{—}1918-1921delGCCT, and combinations thereof.

### List 2

CPT2, p.L7fs, c.20dupT
CPT2, p.T60N, c.179C>A
CPT2_c.233+1G>C
CPT2, p.P227R, c.680C>G
CPT2, p.S267L, c.800C>T
CPT2, p.P284fs, c.848_{—}852delC
CPT2, p.C324Y, c.1025T>C
CPT2, p.M342T, c.1025T>C
CPT2, p.R350C, c.1048C>T
CPT2, p.A367D, c.1100C>A
CPT2, p.L441fs, c.1323_{—}1326delCACT
CPT2, p.K458Q, c.1372A>C
CPT2, p.Q472X, c.1414C>T
CPT2, p.C512Y, c.1535G>A
CPT2, p.T589fs, c.1767delG
CPT2, p.G601R, c.1801G>C
CPT2, p.P604L, c.1811C>T
CPT2, p.E645fs, c.1933-1934insG
ACADVL, p.V164G, c.491T>G
ACADVL, p.delE277, c.827_{—}831del3
PYGM, p.G136fs, c.403_{—}408insG

### List 3

### CPT2 Mutations

| | |
|---|---|
| L7fs, c.20dupT | Truncation mutation |
| P284fs, | Truncation mutation |
| c.848_852delC | |
| L441fs, | Truncation mutation |
| c.1323_1326delCACT | |
| Q472X, c.1414C > T | Truncation mutation |
| T589fs, c.1767delG | Truncation mutation |
| E645fs, c.1933dupG | Truncation mutation |
| c.233 + 1G > C | Splice junction mutation |
| T60N, c.179C > A | PolyPhen2 score = 0.994, probably damaging |
| D118G, c.353A > G | rs148035648 (The G allele was found on one |
| | chromosome of 3832 chromosomes examined |
| | in the ESP cohort population), PolyPhen2 |
| | score = 0.994, probably damaging |
| P227R, c.680C > G | PolyPhen2 score = 1, probably damaging |
| N311S, c.932A > G | rs142790440 (MAF = 0.002 in 4552 |
| | chromosomes examined in the ESP cohort |
| | population), PolyPhen2 |
| | score = 1, probably damaging |
| C324Y, c.971G > A | PolyPhen2 score = 1, probably damaging |
| M342T, c.1025T > C | rs144658100 (MAF = 0.001 in 4552 |
| | chromosomes examined in the ESP cohort |
| | population), PolyPhen2 score = 0.78, possibly |
| | damaging |
| R350C, c.1048C > T | rs151003641 (MAF = 0 in 4552 chromosomes |
| | examined in the ESP cohort population), |
| | PolyPhen2 score = 1, probably damaging |
| A367D, c.1100C > A | PolyPhen2 score = 0.89, possibly damaging |
| A470T, c.1408G > A | PolyPhen2 score = 0.999, probably damaging |
| C512Y, c.1535G > A | PolyPhen2 score = 0.997, probably damaging |
| E545A, c.1634A > C | rs17848485 (MAF = 0.001 in 3220 chromosomes |
| | examined in the ESP cohort population), |
| | PolyPhen2 score = 1, probably damaging |
| G601R, c.1801G > C | PolyPhen2 score = 0.999, probably damaging |
| P604L, c.1811C > T | PolyPhen2 score = 1, probably damaging |

### PYGM Mutations:

| | |
|---|---|
| p.G136fs, c.403_408insG | Truncation mutation |
| p.S277fs, c.830delC | Truncation mutation, rs35091627 |

We further analyzed the List 3 mutations to ascertain actual phenotypes of the individuals from which the samples were obtained. Table 1 summarizes the analysis.

TABLE 1

| | | | Laboratory | Symptom |
|---|---|---|---|---|
| | Infant/Child/Adult | Clinical Features | Features | Triggers |
| CPT2 Mutations | | | | |
| L7fs, c.20dupT | Adult | muscles weak, pain, | CK > 100,000 | exercise, fasting, |
| | | fatigue | | infections |
| P284fs, c.848_852delC | Infant | ND | ND | ND |
| L441fs, c.1323_1326delCACT | Adult | muscle pain, | rhabdomyolysis, elev. | |
| | | weakness | CK, elev. Liver | |
| | | | enzymes | |
| Q472X, c.1414C > T | Adult | muscle pain, | rhabdomyolysis | ND |
| | | weakness, cramps, | | |
| | | fatigue; | | |
| | | cardiomyopathy | | |
| T589fs, c.1767delG | Adult | muscle pain, | | cold weather, |
| | | weakness, fatigue | | viral infection, |
| | | | | stress, |
| | | | | dehydration |
| E645fs, c.1933dupG | Child | muscle pain | ND | ND |
| c.233 + 1G > C | Adult | muscle pain, | rhabdomyolysis, | ND |
| | | cramps | elevated CK | |
| T60N, c.179C > A | Adult | muscle pain, | CK 24,000 | viral infections, |
| | | cramps, weakness, | | exercise, fasting |
| | | stiffness | | |
| D118G, c.353A > G | Child | muscle pain, | diabetes, | |
| | | weakness, cramps, | rhabdomyolysis, elev. | |
| | | fatigue, vomiting | CK | |
| P227R, c.680C > G | Child | ND | ND | ND |
| N311S, c.932A > G | Adult | muscle pain, | elevated CK, | exercise |
| | | weakness | rhabdomyolysis, | |
| | | | abnormal EMG | |
| C324Y, c.971G > A | Adult | muscle pain, | CK 40,000 | exercise, fasting, |
| | | stiffness | | cold temp, |
| | | | | dehydration |
| M342T, c.1025T > C | Infant | ND | ND | ND |
| R350C, c.1048C > T | Adult | no symptoms; son | ND | ND |
| | | is affected | | |
| A367D, c.1100C > A | Adult | muscle pain, | ND | ND |
| | | weakness, cramps, | | |
| | | fatigue; | | |
| | | cardiomyopathy | | |
| A470T, c.1408G > A | Child | muscle pain, | rhabdomyolysis, | ND |
| | | cramps, fatigue | elevated CK | |
| C512Y, c.1535G > A | Child | muscle pain, | ND | exercise, hot |
| | | fasting, vomiting | | weather |
| E545A, c.1634A > C | Child | muscle weakness; | rhabdomyolysis, | exercise, hot |
| | | developmental | elevated CK | weather |
| | | delay | | |
| G601R, c.1801G > C | Infant | hepatitis, cardiac | elevated acylcarnitine | ND |
| | | abnormalities; | | |
| | | abnormal EKG & | | |
| | | MRI | | |
| P604L, c.1811C > T | Adult | muscle pain | rhabdomyolysis | |
| c.1-121C > T | Adult | muscle pain, | rhabdomyolysis | exercise, fasting, |
| | | cramps, weakness, | | lack of sleep, |
| | | stiffness | | cold weather, |
| | | | | stress, |
| | | | | dehydration |
| c.1-117-120del3 | Adult | muscle cramps, | normal EMG, | exercise |
| | | weakness, fstigue, | elevated liver | |
| | | lethargy | enzymes | |
| K79T, c.236A > C | Adult | muscle pain, | normal EMG, | ND |
| | | weakness, | elevated liver | |
| | | progressive course | enzymes | |
| c.341-16T > C | Adult | muscle pain, | normal CK | ND |
| | | weakness, cramps, | | |
| | | fatigue | | |
| S267L, c.800C > T | Adult | muscle pain, | | exercise, fasting, |
| | | cramps, weakness, | | lack of sleep, |
| | | stiffness | | cold weather, |
| | | | | stress, |
| | | | | dehydration |
| K458Q, c.1372A > C | | | | |
| R503R,c.1509C > T | Child | muscle pain, | rhabdomyolysis, | exercise |
| | | weakness, cramps, | elevated CK | |
| | | lethargy | | |
| G526G,c.1578T > C | Adult | muscle cramps, | elevated CK | ND |
| | | weakness, fstigue, | | |
| | | lethargy | | |
| P504P,c.1512G > T | Adult | ND | diabetes, elevated CK | exercise |
| T589T,c.1767G > A | Adult | ND | ND | ND |
| R554X, c. 1660C > T | Adult | ND | ND | ND |
| Two unidentified PYGM | | | | |
| mutations: | | | | |
| p.G136fs, c.403_408insG | Adult | muscle cramps, | elev. CK | statins |
| | | stiffness | | |
| p.S277fs, c.830delC | Adult | muscle pain, | rhabdomyolysis | exercise |
| | | weakness | | |

Key:
CK, serum creatine kinase;
ND, no data

In certain embodiments of the invention, sub-cobminations of the genes in List 1 and/or List 2, and/or List 3 will be a) particularly common either alone or in concert in select groups of individuals tested; and/or b) frequently diagnostic for muscle disease or indicative of risk for muscle disease. The invention thus encompasses individual tests comprising these ensembles and ensemble sub-cobinations of the disease mutations to allow for more individualized testing.

We claim:

1. A method of determining whether an individual is at risk for developing or has a metabolic muscle disorder comprising testing a biological sample obtained from the indivdival for the presence or absence of:
i) a mutation in the CPT2 gene selected from the group of mutations consisting of CPT2P55R_{—}164C>G, CPT2T60N_{—}179C>A, CPT2_ivs2_{—}233+1G>C, CPT2_ivs2_{—}234-1G>A, CPT2C84R_{—}250T>C, CPT2_S86fs_{—}254-257delAG, CPT2S113L_{—}338C>T, CPT2_ivs3_{—}345+5G>A, CPT2Y120C_{—}359A>G, CPT2R124X_{—}370C>T, CPT2R124Q_{—}371G>A, CPT2R151Q_{—}452G>A, CPT2R161W_{—}481C>T, CPT2K164X_{—}490A>T, CPT2_P173S_{—}517C>T, CPT2E174K_{—}520G>A, CPT2L178-I186delinsF_{—}534-558delinsT, CPT2Y210D_{—}628T>G, CPT2D213G_{—}638A>G, CPT2M214T_{—}641T>C, CPT2P227L_{—}680C>T, CPT2P227R_{—}680C>G, CPT2R231W_{—}691C>T, CPT2N250fs_{—}747-749delAA, CPT2_S267L_{—}800C>T, CPT2K274M_{—}821A>T, CPT2P284fs_{—}848-852delC, CPT2R296X_{—}886C>T, CPT2_R296Q_{—}887G>A, CPT2_R296L_{—}887G>T, CPT2_C324Y_{—}971G>A, CPT2_C326Y_{—}977G>A, CPT2D328G_{—}983A>G, CPT2_M342T_{—}1025T>C, CPT2_R350C_{—}1048C>T, CPT2_F352C_{—}1055T>G, CPT2_A367D_{—}1100C>A, CPT2_F383Y_{—}1148T>A, CPT2_S408fs_{—}1221-1224delCT, CPT2Q413fs_{—}1238-1239delAG, CPT2_K414fs_{—}1239-1240delGA, CPT2_T425fs_{—}1273-1274delAC, CPT2_L441fs_{—}1323_{—}1326delCACT, CPT2_F448L_{—}1342T>C, CPT2_R450X_{—}1348A>T, CPT2_E454X_{—}1360G>T, CPT2_K457X_{—}1369A>T, CPT2_K458Q_{—}1372A>C, CPT2_Q472X_{—}1414C>T, CPT2_Y479C_{—}1436A>G, CPT2_G480R_{—}1438G>A, CPT2_T482fs_{—}1444-1447delACAG, CPT2_E487K_{—}1459G>A, CPT2_I502T_{—}1505T>C, CPT2_R503C_{—}1507C>T, CPT2_P504L_{—}1511C>T, CPT2_C512Y_{—}1535G>A, CPT2_A515fs_{—}1543-1546delGCCT, CPT2_F516S_{—}1547T>C, CPT2_H523fs_{—}1567-1570delCA, CPT2_E545del_{—}1631-1636delAAG, CPT2_ivs4_{—}1645+5G>A, CPT2_G549D_{—}1646G>A, CPT2_Q550R_{—}1649A>G, CPT2_D553N_{—}1657G>A, CPT2_R554X_{—}1660C>T, CPT2_R560Q_{—}1679G>A, CPT2_Y579fs_{—}1737delC, CPT2_S588C_{—}1763C>G, CPT2_T589fs_{—}1767delG, CPT2_P595fs_{—}1782-1784delC, CPT2_G600R_{—}1798G>A, CPT2_G601R_{—}1801G>C, CPT2_P604S_{—}1810C>T, CPT2_P604L_{—}1811C>T, CPT2_V605L_{—}1813G>C, CPT2_V606fs_{—}1816-1817delGT, CPT2_D608H_{—}1822G>C, CPT2_Y628S_{—}1883A>C, CPT2_R631C_{—}1891C>T, CPT2_E641fs_{—}1923-1935del13, CPT2_E645fs_{—}1932-1933insA, CPT2_E645fs_{—}1933-1934insG, E645fs, c.1933dupG, N311S, c.932A>G, A470T, c.1408G>A, E545A, c.1634A>C, and combinations thereof, and identifying the individual as at risk for developing having the metabolic muscle disorder wherein the presence of the mutation in the CPT2 gene is determined;
or ii) a mutation in the PYGM gene selected from the group of mutations consisting of PYGM_M1L_{—}1A>C, PYGM_M1V_{—}1A>G, PYGM_L5fs_{—}13-14delCT, PYGM_V16fs_{—}46insTTdelG, PYGM_T26fs_{—}78-79delTG, PYGM_R50X_{—}148C>T, PYGM_Y53X_{—}159C>G, PYGM_Q73fs_{—}212-218dup, PYGM_I83F_{—}247A>T, PYGM_Y85X_{—}255C>A, PYGM_R94W_{—}280C>T, PYGM_N102fs_{—}304-305delA, PYGM_L116P_{—}347T>C, PYGM_E125X_{—}373G>T, PYGM_N134fs_{—}402delC, PYGM_G136fs_{—}403-408insG, PYGM_G136D_{—}407G>A, PYGM_R139W_{—}415C>T, PYGM_G157V_{—}470G>T, PYGM_G159R_{—}475G>A, PYGM_R161C_{—}481C>T, PYGM_K170del_{—}506-511del3, PYGM_G174D_{—}521G>A, PYGM_R194W_{—}580C>T, PYGM_G205S_{—}613G>A, PYGM_P230R_{—}689C>G, PYGM_V239de1_{—}715-717delGTC, PYGM_ivs6_{—}773-2A>T, PYGM_R270X_{—}808C>T, PYGM_ivs7_{—}855+1G>C, PYGM_L292P_{—}875T>C, PYGM_Q337R_{—}1010A>G, PYGM_E349K_{—}1045G>A, PYGM_E349X_{—}1045G>T, PYGM_W362X_{—}1085G>A, PYGM_ivs9_{—}1092+1G>A, PYGM_ivs9_{—}1093-1G>T, PYGM_A365E_{—}1094C>A, PYGM_A365V_{—}1094C>T, PYGM_T379M_{—}1136C>T, PYGM_E383K_{—}1147G>A, PYGM_A384D_{—}1151C>A, PYGM_L385fs_{—}1155-1156delGG, PYGM_W388fs_{—}1162insAdel8, PYGM_L397P_{—}1190T>C, PYGM_ivs10_{—}1239+1G>A, PYGM_R428C_{—}1282C>T, PYGM_G449R_{—}1345G>A, PYGM_S450L_{—}1349C>T, PYGM_A452fs_{—}1354insC, PYGM_V456M_{—}1363G>A, PYGM_G486D_{—}1457G>A, PYGM_T488N_{—}1463C>A, PYGM_T488I_{—}1463C>T, PYGM_R490W_{—}1468C>T, PYGM_R490Q_{—}1469G>A, PYGM_R491Afs_{—}1469-1470dupG, PYGM_R491C_{—}1471C>T, PYGM_W492X_{—}1475G>A, PYGM_V494fs_{—}1479-1480delG, PYGM_D511fs_{—}1530-1531delG, PYGM_D534fs_{—}1601delA, PYGM_E541X_{—}1621G>T, PYGM_K543X_{—}1627A>T, PYGM_K543T_{—}1628A>C, PYGM_R570W_{—}1708C>T, PYGM_R570Q_{—}1709G>A, PYGM_Y574X_{—}1722T>G, PYGM_K575E_{—}1723A>G, PYGM_R576X_{—}1726C>T, PYGM_Q577R_{—}1730A>G, PYGM_L587P_{—}1760T>C, PYGM_ivs14_{—}1768+1G>A, PYGM_R590H_{—}1769G>A, PYGM_F599fs_{—}1792-1797delT, PYGM_R602W_{—}1804C>T, PYGM_R602Q_{—}1805G>A, PYGM_ivs15_{—}1828-1G>A, PYGM_R650X_{—}1948C>T, PYGM_E655K_{—}1963G>A, PYGM_A660D_{—}1979C>A, PYGM_D662A_{—}1985A>C, PYGM_Q666E_{—}1996C>G, PYGM_N685Y_{—}2053A>T, PYGM_G686R_{—}2056G>A, PYGM_G686R_{—}2056G>C, PYGM_A687P_{—}2059G>C, PYGM_T692fs_{—}2075insAAAdelCC, PYGM_A704V_{—}2111C>T, PYGM_G705fs_{—}2112-2114delGG, PYGM_F709-F710del_{—}2125-2130delTTC, PYGM_R715W_{—}2143C>T, PYGM_K754fs_{—}2260-2262delA, PYGM_Q755X_{—}2263C>T, PYGM_ivs18_{—}2312+3G>C, PYGM_C784X_{—}2352C>A, PYGM_ivs19_{—}2380-1G>A, PYGM_P795fs_{—}2385-2386delAA, PYGM_W798R_{—}2392T>C, PYGM_D815A_{—}2444A>C, PYGM_W826S_{—}2477G>C, p.S277fs, c.830delC, and combinations thereof, and identifying the individual as at risk for developing having the metabolic muscle disorder wherein the presence of the mutation in the PYGM gene is determined.

2. The method of claim 1, wherein the mutation in the CPT2 gene is determined, and wherein the indivdival is identified as at risk for or having CPT2 deficiency.

3. The method of claim 2, wherein the mutation in the CPTR gene comprises one or more of L7fs, c.20dupT, P284fs, c.848_{—}852delC, L441fs, c.1323_{—}1326delCACT, Q472X, c.1414C>T, T589fs, c.1767delG, E645fs, c.1933dupG, c.233+1G>C, T60N, c.179C>A, D118G, c.353A>G, P227R, c.680C>G, N311S, c.932A>G, C324Y, c.971G>A, M342T, c.1025T>C, R350C, c.1048C>T, A367D, c.1100C>A, A470T, c.1408G>A, C512Y, c.1535G>A, E545A, c.1634A>C, G601R, c.1801G>C, P604L, c.1811C>T, c.1-121C>T, c.1-117-120del3, K79T, c.236A>C, c.341-16T>C, S267L, c.800C>T, K458Q, c.1372A>C, R503R, c.1509C>T, G526G, c.1578T>C, P504P, c.1512G>T, T589T, c.1767G>A, and R554X, c.1660C>T.

4. The method of claim 3, wherein the mutation in the CPT2 gene is comprises one or more of E645fs, c.1933dupG, N311S, c.932A>G, A470T, c.1408G>A and E545A, c.1634A>C.

5. The method of claim 1, wherein the mutation in the PYGM gene is determined, and wherein the indivdival is identified as at risk for or having McArdle disease.

6. The method of claim 4, wherein the mutation in the PYGM gene comprises one more more of p.G136fs, c.403_{—}408insG and p.S277fs, c.830delC.

7. The method of claim 1, wherein the presence or absence of at least 20 mutations the CPT2 gene and/or the PYGM gene are determined

8. The method of claim 7, wherein the presence or absence of at least 50 mutations the CPT2 gene and/or the PYGM gene are determined

9. The method of claim 1, wherein the sample is obtained from an adult.

10. The method of claim 1, wherein the sample is obtained from a child.
